SEQUENCE LISTING (1 sequence)

SEQ ID NO: 1 - DNA, 1524 bp, Artificial Sequence
  Recombinant DNA made in the lab
atgggatggtcatgtatcatcctttttctagtagcaactgcaaccggtgtacattccgacatccagatgacccagtctccatccgctctgtctgcatccgttggagacagagttaccatcacttgccatgccagtcaaaacatttatgtttggttaaactggtaccagcagaaaccaggaaaagcccctaaactcttgatctataaggcttccaacctgcacacaggcgtcccatcaaggtttagtggcagtggatctggaacagacttcacattaaccatcagcagcctgcagcctgaagactttgccacttactactgtcaacagggtcaaacttatccgtacacgttcggaggggggaccaaggtggaaataaaaggaggaggaggaagccaggtccaactggtgcagtccggagctgaggtgaagaagccgggggcttcagtgaaggtttcctgcaaggcttctggctacaccttcacaagctactatatacactgggtgagacaggcgcctggacagggacttgagtggattggatgtatttatcctggaaatgtcaatactaactataatgagaagttcaaggacagggccacactgactgtagacacatccatcagcactgcctacatggagctcagcagactgcgctctgacgacactgcggtctatttctgtacaagatcacactacggcctcgactggaacttcgatgtctggggccaagggaccacggtcaccgtctcctcaggtggtggtggtagtgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagtcgaaggtggaagtggaggttctggtggaagtggaggttcaggtggagtcgacgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaagaattccatcatcaccaccatcactga

(19) United States
(12) Patent Application Publication
(10) Pub. No.: US 2021/0032337 A1
(43) Pub. Date: Feb. 4, 2021

(54) METHOD FOR EFFICIENTLY ACTIVATING AND EXPANDING T CELLS

(71) Applicant: Wenyun Zhou, San Diego, CA (US)

(72) Inventor: Wenyun Zhou, San Diego, CA (US)

(21) Appl. No.: 16/531,101

(22) Filed: Aug. 4, 2019

(51) Int. Cl. C07K 16/28; A61K 35/17; C12N 5/0783

(52) U.S. Cl. CPC C07K 16/2809; A61K 35/17; C12N 5/0636; C07K 16/2818; C07K 2317/24; C07K 16/2875; C07K 16/2827; C07K 2317/31; C07K 16/2878

(57) ABSTRACT

The present invention discloses that T cells can be efficiently activated and expanded by a simple monomeric single chain bispecific antibody without using complicated beads, accessory cells, antibody-coating plates, or tetrameric antibody complex. The present invention also discloses that T cells activated and expanded by the monomeric single chain bispecific antibody can be efficiently transduced by virus.

### FIELD OF THE INVENTION

The present invention relates to the field of T cell immunotherapy. In particular, embodiments of the invention relate to the non-accessory cells, non-antibody coating and non-beads method for efficiently activating and expanding T cells used in cell immunotherapy.

### BACKGROUND OF THE INVENTION

T cell immunotherapy methods have been developed in order to enhance the host immune response to tumors and viruses (Kamphorst A O, 2013). In fact, the adoptive transfer of T cells expressing genetically engineered chimeric antigen receptors (CARs) has shown great promise in clinical studies addressing chronic or acute lymphocytic leukemia (Kalos, M, 2011; Maus, M. V, 2014). T cell immunotherapy methods often involve the ex-vivo activation and expansion of T cells. T cell activation is required for the efficient transduction of the CAR cDNA via retroviral vectors (Xiuyan Wang, 2016). One of the challenges of T cell immunotherapy is the development of efficient technologies and cost-effective manufacturing platforms for activating and expanding T cells.

Currently there are several methods for activating and expanding T cells: i) Dendritic cells (DCs) are the endogenous activators of T cells (Petra Kleindienst, 2003). While therapeutic applications of DCs continue to be investigated, DC potency varies from patient to patient. Such limitation hampers the usage of DCs as a reliable source for T-cell activation; ii) Another cell-based T cell activation approach is through artificial antigen-presenting cells (AAPCs). Irradiated K562-derived AAPCs have been used to stimulate the expansion of T cells (T Numbenjapon, 2006). The generation and selection of GMP-grade HLA-matched AAPC line is complex and requires additional resources (Singh H, 2014); iii) T cells can be activated and expanded by coating plates with anti-CD3 antibody and anti-CD28 antibody (Walker M R, 2003). However, the antibody coating efficiency could vary dramatically; iiii) Beads based T cell activation methods have also been developed. Several biotech companies have generated beads based T cell activation reagents including Gibco™ Dynabeads™ Human T-Activator CD3/CD28 (Cat #111.61D, Thermo Fisher Scientific) and Miltenyi MACS GMP TransAct CD3/28 beads (Cat #130-020-008, Miltenyi Biotech). The use of beads with immobilized mAbs for expansion of T cells in cell therapy protocols requires the separation and removal of the beads from the T cells prior to patient infusion. This process could greatly reduce the quantity of beads remaining with the T cells, but does not completely eliminate the beads. This incomplete bead removal results in some beads being infused in patients which could cause toxic effects. The magnetic bead removal process also reduces the number of T cells available for therapy, as many T cells remain associated with the paramagnetic beads even after the waiting time and mechanical disassociation. Miltenyi TransAct CD3/CD28 beads are bio-degradable, but the generation and selection of GMP-grade beads is complex and expensive. The complicated manufacturing process needs to be simplified to promote standardization. The in vivo use of bio-degradable TransAct CD3/28 beads will also face many regulation challenges; inn) A US patent application (US 20070036783A1) reports using a soluble complex of to activate and expand T cells. This complex is a tetrameric antibody complex (TAC). Manufacturing process of tetrameric antibody complex is complicated. Components of tetrameric antibody complex are various among different batches.

The above limitations can be overcome with using the monomeric single chain bispecific antibody to activate and expand T cells. The monomeric single chain bispecific antibody activates T cells through the simultaneous engagement of the primary signal and the co-stimulatory signal. The monomeric single chain bispecific antibody used in the present invention has important attributes that make it attractive from the manufacturing and regulatory standpoint. The structure of monomeric single chain bispecific antibody molecule is simple as disclosed in FIG. 1 in the present invention. It is a soluble and component defined reagent. It is a monomer reagent. The monomeric single chain bispecific antibody is highly suitable for use in aseptic cell manufacturing as it can be sterile filtered. T cells expanded by the monomeric single chain bispecific antibody can be efficiently transduced by lentivirus and can perform cytotoxic effects as disclosed in the present invention.

### SUMMARY OF THE INVENTION

The present invention discloses that a monomeric single chain bispecific antibody peptide, which is constructed by simply connecting two T cell antigen binding domains (such as anti-CD28 scfv and anti-CD3 scfv) with several amino acids spacer, can efficiently activating and expanding T cells. The present invention simplifies the procedures of T cell activation and expansion and lowers the cost for generating large amount of T cells used in cell immunotherapy.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 showed the structure of the monomeric single chain bispecific antibody. FIG. 1A showed the general structure of monomeric single chain bispecific antibody, which was composed of: first antigen binding domain, linker, second antigen binding domain, linker and Tag which can be used in antibody detection and purification. FIG. 1B showed the structure of anti-CD28/CD3 monomeric single chain bispecific antibody, as one example of monomeric single chain bispecific antibodies. The anti-CD28/CD3 monomeric single chain bispecific antibody was composed of: anti-CD28 antibody light chain variable domain, linker, anti-CD28 antibody heavy chain variable domain, linker, anti-CD3 antibody heavy chain variable domain, linker, anti-CD28 antibody light chain variable domain and Tag. In FIG. 1, letter H represents antibody heavy chain variable domain; letter L represents antibody light chain variable domain.

FIG. 2 showed the binding activities of anti-CD28CH2CH3 antibody and anti-CD3CH2CH3 antibody. FIG. 2A indicated that anti-CD28CH2CH3 was functional antibody. FIG. 2B indicated that anti-CD3CH2CH3 antibody was functional antibody. Solid line: Jurkat cells were first stained with anti-CD28CH2CH3 antibody, or anti-CD3CH2CH3 antibody, then stained with PE-conjugated anti-human IgG FC antibody. Dashed line: Jurkat cells were only stained with PE-conjugated anti-human IgG FC antibody. Both anti-CD28CH2CH3 antibody and anti-CD3CH2CH3 antibody were used to confirm the binding specificities of anti-CD28/CD3 monomeric single chain bispecific antibody.

FIG. 3 proved that the monomeric single chain anti-CD28/CD3 bispecific antibody had two binding parts, one binding to CD28, the other binding to CD3. FIG. 3A showed that anti-CD28/CD3 antibody still could bind to Jurkat cells after only blocking CD28 binding site with anti-CD28CH2CH3 antibody, which proved that anti-CD28/CD3 bispecific antibody had binding ability to CD3. FIG. 3B showed that anti-CD28/CD3 antibody still could bind to Jurkat cells after only blocking CD3 binding site with anti-CD3CH2CH3 antibody, which proved that anti-CD28/CD3 bispecific antibody had binding ability to CD28. Solid line: Jurkat cells were first blocked with anti-CD28CH2CH3 antibody, or anti-CD3CH2CH3 antibody, secondly stained with anti-CD28/CD3 antibody, lastly stained with PE-conjugated anti-His antibody. Dashed line: Jurkat cells were only stained with PE-conjugated anti-His antibody.

FIG. 4 showed that T cells can be efficiently activated by the monomeric single chain anti-CD28/CD3 bispecific antibody according to the expression of T cells activation marker CD25. T cells were incubated with anti-CD28/CD3 bispecific antibody supernatant for 15 minutes, washed twice and then cultured for three days. 2e6 anti-CD28/CD3 bispecific antibody treated T cells were stained with CD25FITC antibody or control antibody. FIG. 4A showed the FACS result of activated T cells stained with control antibody. FIG. 4B showed the FACS result of activated T cells stained with FITC-conjugated anti-CD25 antibody. 87.1% T cells expressed CD25 after stimulated by monomeric single chain anti-CD28/CD3 bispecific antibody.

FIG. 5 showed that T cells can be efficiently expanded by the monomeric single chain anti-CD28/CD3 bispecific antibody according to the 16 days cell number counting.

FIG. 6 showed that T cells activated with monomeric single chain anti-CD28/CD3 bispecific antibody were efficiently transduced by RFP lentivirus. 2e6 anti-CD28/CD3 bispecific antibody activated T cells were infected with 20 ul, 40 ul, 80 ul RFP lentivirus supernatant. Infection efficiency was analyzed by FACS. FIG. 6A showed the transduction result with 0 ul RFP virus. FIG. 6B showed the transduction result with 20 ul RFP virus. FIG. 6C showed the transduction result with 40 ul RFP virus. FIG. 6D showed the transduction result with 80 ul RFP virus.

### DEFINITIONS

Unless defined otherwise, all technical and scientific terms used herein have the same meaning as commonly understood by one of ordinary skill in the art to which the invention pertains. It is also to be understood that the terminology used herein is for the purpose of describing particular embodiments only, and is not intended to be limiting.

The term “antibody” as used herein, refers to an immunoglobulin molecule which specifically binds with an antigen. Antibodies can be intact immunoglobulins derived from natural sources or from recombinant sources and can be immunoreactive portions of intact immunoglobulins. The antibody in the present invention may exist in a variety of forms where the antigen binding portion of the antibody is expressed as part of a contiguous polypeptide chain including, for example, a single domain antibody fragment (sdAb), a single chain antibody (scfv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.).

The term “bispecifc antibody” as used herein, refers to a type of antibody that can bind to two different antigens.

As used herein, a “single chain variable fragment (scfv)” means a single chain polypeptide derived from an antibody which retains the ability to bind to an antigen. An example of the scfv includes an antibody polypeptide which is formed by a recombinant DNA technique and in which Fv regions of immunoglobulin heavy chain (H chain) and light chain (L chain) fragments are linked via a spacer sequence.

As used herein, the term “antigen binding domain” refers to any antigen binding polypeptide, a wide variety of which are known in the art. The antigen binding domain can be any domain that binds to the antigen. In some embodiments, the antigen binding domain is a single chain Fv (scfv). In some embodiments, other antibody based recognition domains (cAb VHH (camelid antibody variable domains) and humanized versions, IgNAR VH (shark antibody variable domains) and humanized versions, sdAb VH (single domain antibody variable domains) and “camelized” antibody variable domains are suitable for use. In some embodiment, T cell receptor (TCR) based recognition domain such as single chain TCR (scTv, single chain two-domain TCR containing V.alpha.V.beta.) is also suitable for use. In some embodiments, the antigen binding domain may be chosen to recognize a ligand that acts as a cell surface marker on target cells associated with a particular disease state. In some embodiments, the antigen binding domain could be an alternative scaffold known in the art to function as binding, such as a recombinant fibronectin domain, and the like.

The term “T cell” refers to all types of immune cells expressing CD3 including T helper cells, cytotoxic T cells, T regulatory cells and gamma-delta T cells.

### DETAILED DESCRIPTION OF THE INVENTION

Terms and symbols of genetics, molecular biology, biochemistry and nucleic acid used herein follow those of standard treatises and texts in the field, e.g. Kornberg and Baker, DNA Replication, Second Edition (W.H. Freeman, New York, 1992); Lehninger, Biochemistry, Second Edition (Worth Publishers, New York, 1975); Strachan and Read, Human Molecular Genetics, Second Edition (Wiley-Liss, New York, 1999). All terms are to be understood with their typical meanings established in the relevant art.

T cells require at least two signals to become fully activated (Geppert T D, 1990; Peter A. Bretscher, 1999; Bretscher P, 1970). The first occurs after engagement of the T cell antigen-specific receptor (TCR), and the second occurs after subsequent engagement of co-stimulatory molecules. The present invention discloses a method through using the monomeric single chain bispecific antibody to activate and expand T cells. The monomeric single chain bispecific antibody comprises two antigen binding domains on a single polypeptide chain, wherein one of the antigen binding domains provides primary stimulatory signal, and the other antigen binding domain provides co-stimulatory signal. The single polypeptide nature of the bispecific antibody eliminates the need to employ a process to separate and remove the beads from the T cells prior to infusion into a patient. The monomeric single chain bispecific antibody comprises two activation signals, which eliminates the need to coat antibodies to solid surface, or use accessory cell for activating T cells.

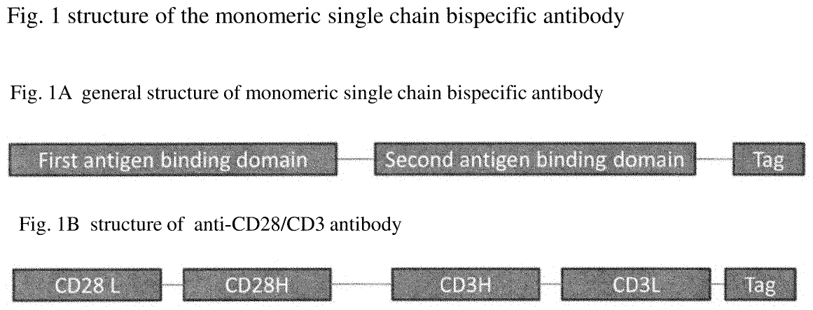

The monomeric single chain bispecific antibody is constructed by unifying two antigen binding domains of different specificity into a single construct. Two antigen binding domains are linked via a short polypeptide chain. The general structure of the monomeric single chain bispecific antibody was shown in the present invention in FIG. 1A.

In one embodiment, a member of the antigen binding domain in the monomeric single chain bispecific antibody is a single chain Fv (scfv).

In one embodiment, a member of the antigen binding domain in the monomeric single chain bispecific antibody is a single chain TCR (scTv).

In one embodiment, a member of the antigen binding domain in the monomeric single chain bispecific antibody suitable for use in a subject is a ligand for a receptor, or a receptor-binding fragment of a ligand.

In one embodiment, a member of the antigen binding domain in the monomeric single chain bispecific antibody is derived from an antibody against CD3, or the CD3-binding portion thereof. This antigen binding domain provides T cells primary stimulatory signal. This antigen binding domain may be from a hybridoma clone, and may be a humanized form of a non-human antibody. In one embodiment, this antigen binding domain is derived from OKT3 or G19-4 or the CD3-binding portion thereof.

In one embodiment, a member of the antigen binding domain in the monomeric single chain bispecific antibody is derived from an antibody against CD2, or the CD2-binding portion thereof. This antigen binding domain provides T cells primary stimulatory signal. This antigen binding domain may be from a hybridoma clone, and may be a humanized form of a non-human antibody.

In one embodiment, a member of the antigen binding domain in the monomeric single chain bispecific antibody is derived from an antibody against CD28, or the CD28-binding portion thereof. This antigen binding domain provides T cells co-stimulatory signal. This antigen binding domain may be from a hybridoma clone, and may be a humanized form of a non-human antibody. In one embodiment, this antigen binding domain is derived from EX5.3D10, or the CD28-binding portion thereof.

In one embodiment, a member of the antigen binding domain in the monomeric single chain bispecific antibody is derived from an antibody against CD9, or the CD9-binding portion thereof. This antigen binding domain provides T cells co-stimulatory signal. This antigen binding domain may be from a hybridoma clone, and may be a humanized form of a non-human antibody. In one embodiment, this antigen binding domain is derived from ES5.2D8 or the CD9-binding portion thereof.

In one embodiment, a member of the antigen binding domain in the monomeric single chain bispecific antibody is derived from an antibody against CD137, or the CD137-binding portion thereof. This antigen binding domain provides T cells co-stimulatory signal. This antigen binding domain may be from a hybridoma clone, and may be a humanized form of a non-human antibody.

In one embodiment, a member of the antigen binding domain in the monomeric single chain bispecific antibody is derived from an antibody against ICOS, or the ICOS-binding portion thereof. This antigen binding domain provides T cells co-stimulatory signal. This antigen binding domain may be from a hybridoma clone, and may be a humanized form of a non-human antibody.

In one embodiment, a member of the antigen binding domain in the monomeric single chain bispecific antibody is derived from an antibody against CD40, or the CD40-binding portion thereof. This antigen binding domain provides T cells co-stimulatory signal. This antigen binding domain may be from a hybridoma clone, and may be a humanized form of a non-human antibody.

In some embodiments, the antigen binding domains of the monomeric single chain bispecific antibody may be arranged, as either protein N terminal-(antigen binding domain for primary stimulatory signal)-(antigen binding domain for co-stimulatory signal)-C terminal or protein N terminal-(antigen binding domain for co-stimulatory signal)-(antigen binding domain for primary stimulatory signal)-C terminal. In one embodiment, the first and/or second domains of the monomeric single chain bispecific antibody are/is independently derived from an antibody produced in primate, rodent, tylopoda or cartilaginous fish. The first and/or second domain of the monomeric single chain bispecific antibody may be either naturally occurring or genetically engineered.

In one embodiment, the monomeric single chain bispecific antibody is derived from mouse anti-antigen antibodies or fragments thereof. In another embodiment, the monomeric single chain bispecific antibody is derived from humanized anti-antigen antibodies or fragments thereof. In yet another embodiment, the monomeric single chain bispecific antibody is derived from fully human anti-antigen antibodies or fragments thereof.

In some embodiments, it is beneficial for the antigen binding domain to be derived from the same species in which the monomeric single chain bispecific antibody modified T cells will ultimately be used in. For example, for use in human, it may be beneficial for the monomeric single chain bispecific antibody to be derived from human.

According to a further embodiment of the invention, the monomeric single chain bispecific antibody may be subjected to an alteration to render it less immunogenic when administered to a human. One of ordinary skill in the art will understand how to determine whether, and to what degree an antibody must be altered in order to prevent it from eliciting an unwanted host immune response.

In some embodiments, the monomeric single chain bispecific antibody construct further comprises a domain which facilitates its purification and detection, for examples: His-tag, HA-tag, GST-tag, CH2CH3-tag, and the like.

In some embodiments, the monomeric single chain bispecific antibody can be purified by many techniques well known in the art, such as reverse phase chromatography, high performance liquid chromatography (HPLC), ion exchange chromatography, size exclusion chromatography, affinity chromatography, gel electrophoresis, and the like. The actual conditions used to purify the monomeric single chain bispecific antibody will depend, in part, on production strategy and on factors such as net charge, hydrophobicity, hydrophilicity, and the like, and will be apparent to those of ordinary skill in the art. For affinity chromatography purification, any antibody which specifically binds the single chain bispecific antibody, or an affinity tag attached thereto, may for example be used.

In some embodiments, the T cells activated and expanded by the monomeric single chain bispecific antibody may be derived from T cell lineage; The T cells activated and expanded by the monomeric single chain bispecific antibody may be mature T cells; The T cells activated and expanded by the monomeric single chain bispecific antibody may be precursor T cells; The T cells activated and expanded by the monomeric single chain bispecific antibody may be cytotoxic T cells; The T cells activated and expanded by the monomeric single chain bispecific antibody may be naive T cells; The T cells activated and expanded by the monomeric single chain bispecific antibody may be memory stem cell T cells (TMsc); The T cells activated and expanded by the monomeric single chain bispecific antibody may be central memory T cells (TCM); The T cells activated and expanded by the monomeric single chain bispecific antibody may be effector T cells (TE); The T cells activated and expanded by the monomeric single chain bispecific antibody may be CD4+ T cells; The T cells activated and expanded by the monomeric single chain bispecific antibody may be CD8+ T cells; The T cells activated and expanded by the monomeric single chain bispecific antibody may be CD4+ and CD8+ cells; The T cells activated and expanded by the monomeric single chain bispecific antibody may be alpha-beta T cells; The T cells activated and expanded by the monomeric single chain bispecific antibody may be gamma-deta T cells; The T cells activated and expanded by the monomeric single chain bispecific antibody may be natural killer T cells; The T cells activated and expanded by the monomeric single chain bispecific antibody may be helper T cells; The T cells activated and expanded by the monomeric single chain bispecific antibody may be regulatory T cells (Treg).

In some embodiments, T cells activated and expanded by the monomeric single chain bispecific antibody may be obtained from a patient, or may be obtained from an existing culture of T cells. The T cells activated and expanded by the monomeric single chain bispecific antibody can be obtained from, e.g., peripheral blood, placental blood, umbilical cord blood, bone marrow, lymph node tissue, spleen tissue, tumor tissue, or the like. T cells from peripheral blood can be separated, e.g., by leukopheresis. Specific subpopulations of T cells, such as CD4+, CD8+, or any of the other T cell populations, may be isolated from the leukocytes by positive and/or negative selection techniques. Appropriate combinations of antibodies for either positive or negative selection will be apparent to those of skill in the art.

In some embodiments, the T cells can be incubated with the monomeric single chain bispecific antibody for 15 minutes in a sterile tube, washed twice by PBS, then cultured in non-tissue culture treated vessels. Expansion of T cells may proceed for, e.g., 1, 2, 3, 4, 5, 6, 7, 8, 9, 10, 11, 12, 13, 14, 15, 16, 17, 18, 19, 20, 21 days or more. The degree of expansion may be analyzed at one or more time points during expansion by, e.g., counting a sample of cells with a cell counter, flow cytometry, or the like.

In one embodiment, the T cells can be expanded with the monomeric single chain bispecific antibody from about 10-fold to about 1,000-fold.

The present invention utilizes the monomeric single chain anti-CD28/CD3 bispecific antibody, as the example, to show the non-beads, non-accessory cells and non-coating method for activating and expanding T cells. The monomeric single chain anti-CD28/CD3 bispecific antibody comprises two antigen binding domains, wherein a portion of the bispecific antibody is capable of binding to human CD3 surface antigen (primary signaling protein), and another portion of the bispecific antibody is capable of binding to human CD28 antigen (co-stimulatory signaling protein). The sequence of the monomeric single chain anti-CD28/CD3 bispecific antibody was listed in the present invention as SEQUENCE ID NO 1. The structure of the monomeric single chain anti-CD28/CD3 bispecific antibody was disclosed in the present invention in FIG. 1B.

Benefits of using the monomeric single chain bispecific antibody to activate and expand T cells: i) Ease-of-use: no need to prepare autologous APC, feeder cells, or antigens, no need to pre-coat antibodies to all kind of beads or tissue culture plates, no need to separate activated T cells from activating reagents such as Dynabeads™ Human T-Activator CD3/CD28; ii) Physiological activation: Simultaneous stimulation with primary signal and secondary signal; iii) It is an efficient technology and cost-effective manufacturing platform for activating and expanding T cells.

### Examples

The following examples are included to demonstrate preferred embodiments of the invention. It should be appreciated by those of skill in the art that the techniques disclosed in the examples, which follow represent techniques discovered by the inventor to function well in the practice of the invention, and thus can be considered to constitute preferred modes for its practice. However, those of skill in the art should, in light of the present invention, appreciate that many changes can be made in the specific embodiments which are disclosed and still obtain a like or similar result without departing from the spirit and scope of the invention.

### Plasmid DNA Constructs

The plasmid DNA construct of monomeric single chain anti-CD28/CD3 bispecific antibody expression (named as Pcdna3-anti-CD28/CD3) described in the present invention were carried out by local biotech company according to the general techniques of genetic engineering and molecular cloning detailed in Molecular Cloning, A Laboratory Manual, 3rd edition, Cold Spring Harbour Laboratory Press, Cold Spring Harbour, N.Y. (2001). The monomeric single chain anti-CD28/CD3 bispecific antibody contained the following elements: anti-human CD28VL; 5 amino acid linker of sequence; anti-human CD28 VH; 5 amino acid spacer of sequence; anti-human CD3 VH; 18 amino acid spacer of sequence; anti-human CD3 VL; 6 His amino acid Tag. The sequence was listed in SEQUENCE ID NO 1 in the present invention. The monomeric single chain anti-CD28/CD3 bispecific antibody gene was synthesized by local biotech company and cloned into plasmid vector Pcdna3.1+(V79020, Invitrogen, Carlsbad, Calif.).

The plasmid DNA construct of anti-CD28CH2CH3 antibody expression (named as Pcdna3-anti-CD28CH2CH3, CH2CH3 is human IgG1 constant region 2 and 3) described in the present invention were carried out according to the general techniques of genetic engineering and molecular cloning detailed in Molecular Cloning, A Laboratory Manual, 3rd edition, Cold Spring Harbour Laboratory Press, Cold Spring Harbour, N.Y. (2001). The anti-CD28CH2CH3 antibody contained the following elements: anti-human CD28VL; 5 amino acid linker of sequence; anti-human CD28 VH; CH2CH3 fragment. CH2CH3 fragment was used as a tag for purification and detection. The sequence was derived from SEQUENCE ID NO 1 as disclosed in the present invention. The anti-CD28CH2CH3 antibody gene was synthesized by local biotech company and cloned into plasmid vector Pcdna3.1+(V79020, Invitrogen, Carlsbad, Calif.). Anti-CD28CH2CH3 antibody was used as a CD28 binding blocker for verifying the binding activities of monomeric single chain anti-CD28/CD3 bispecific antibody.

The plasmid DNA construct of anti-CD3CH2CH3 antibody expression (named as Pcdna3-anti-CD3CH2CH3) described in the present invention were carried out according to the general techniques of genetic engineering and molecular cloning detailed in Molecular Cloning, A Laboratory Manual, 3rd edition, Cold Spring Harbour Laboratory Press, Cold Spring Harbour, N.Y. (2001). The anti-CD3CH2CH3 antibody contained the following elements: anti-human CD3 VH; 18 amino acid spacer of sequence; anti-human CD3 VL; CH2CH3 fragment. CH2CH3 fragment was used as a tag for purification and detection. The sequence was derived from SEQUENCE ID NO 1 as disclosed in the present invention. The anti-CD3CH2CH3 gene was synthesized by local biotech company and cloned into plasmid vector Pcdna3.1+(V79020, Invitrogen, Carlsbad, Calif.). Anti-CD3CH2CH3 antibody was used as a CD3 binding blocker for verifying the binding activities of the monomeric single chain anti-CD28/CD3 bispecific antibody.

### Protein Production

The monomeric single chain anti-CD28/CD3 bispecific antibody, anti-CD28CH2CH3 antibody, and anti-CD3CH2CH3 antibody described in the present invention were produced by FreeStyle™ 293 Expression System (K900001, Invitrogen, Carlsbad, Calif.) according to the manufacturer's protocol. Briefly, the sequence verified plasmids were used to transfect free FreeStyle™ 293 cells. Transfected free FreeStyle™ 293 cells were cultivated in serum-free FreeStyle™ 293 Expression Medium, in an incubator at 37° C., 95% humidity and 8% CO2. Transfection was performed using the FreeStyle™ MAX Reagent (Ser. No. 16/447,100, Invitrogen, Carlsbad, Calif.) according to the manufacturer's protocol. Transfected cells were incubated for 7 days on an orbital shaker platform rotating at 135 rpm. The anti-CD28CH2CH3 antibody and anti-CD3CH2CH3 antibody were purified by protein A agarose (Cat #20333, Thermo Fisher) according to the manufacturer's protocol. The monomeric single chain anti-CD28/CD3 bispecific antibody was harvested by directly collecting supernatant. The supernatant was aliquoted and stored at −80° C.

### Characterization of Anti-CD28CH2CH3 Antibody and Anti-CD3CH2CH3 Antibody

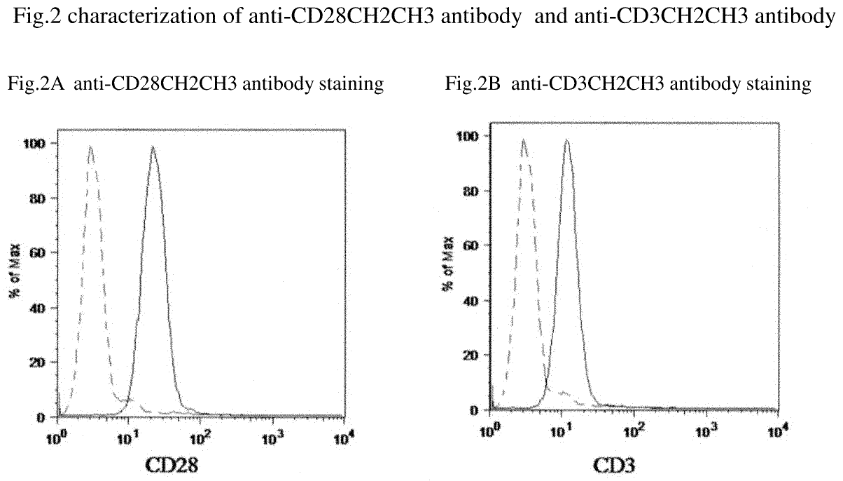

Binding activities of anti-CD28CH2CH3 antibody and anti-CD3CH2CH3 antibody were shown in FIG. 2. 2×10e6 Jurkat T cells (ATCC TIB-152, a human T lymphocyte cell line which expresses both human CD28 and human CD3 protein on the cell surface) were stained with 50 ng anti-CD28CH2CH3 antibody or 50 ng anti-CD3CH2CH3 antibody in FACS buffer (PBS with 1% FBS) for 20 minutes, washed three times with FACS buffer, stained with anti-human IgG Fc PE-conjugated antibody (FAB110P-100, R&D Systems) in FACS buffer for 20 minutes, washed three times with FACS buffer and then subjected for FACS analysis. Flow cytometry was performed on a FACS-Calibur apparatus. The CellQuest software was used to acquire the data (BD BioSciences, Mountain View, Calif.). All flow cytometry data were analyzed with FlowJo software (TreeStar, San Carlos, Calif.). As shown in FIG. 2, both anti-CD28CH2CH3 antibody and anti-CD3CH2CH3 antibody had strong binding activities to Jurkat T cells.

### Characterization of the Monomeric Single Chain Anti-CD28/CD3 Bispecific Antibody

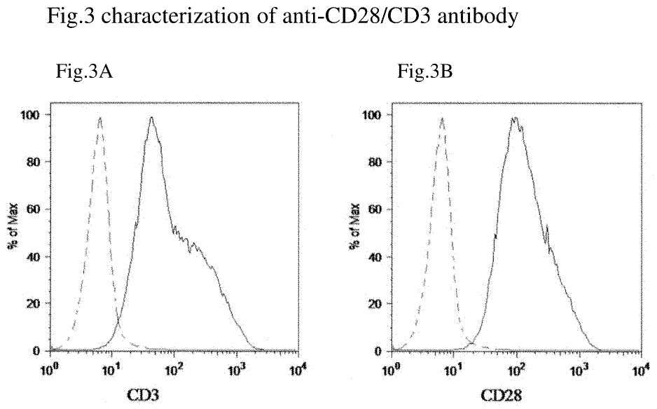

Binding specificity of the monomeric single chain anti-CD28/CD3 bispecific antibody to CD3 was shown by flow cytometric analysis on Jurkat cells. Jurkat cells were cultured in complete RPMI 1640 with 10% FCS (GIBCO). The Jurkat cells were first blocked with 2 ug anti-CD28CH2CH3 antibody for 30 min at 4° C. in FACS buffer, washed twice with FACS buffer, incubated with 50 ul monomeric single chain anti-CD28/CD3 bispecific antibody supernatant for 30 min at 4° C. in FACS buffer, washed twice with FACS buffer, stained with anti-His Tag PE-conjugated Antibody (R&D System, cat #IC050P), washed three times with FACS buffer and then subjected for FACS analysis. Flow cytometry was performed on a FACS-Calibur apparatus; the CellQuest software was used to acquire and analyze the data (BD BioSciences, Mountain View, Calif.). As shown in FIG. 3A, monomeric single chain anti-CD28/CD3 bispecific antibody had specific binding activities to CD3.

Binding specificity of the monomeric single chain anti-CD28/CD3 bispecific antibody to CD28 was shown by flow cytometric analysis on Jurkat cells. Jurkat cells were cultured in complete RPMI 1640 with 10% FCS (GIBCO). The Jurkat cells were first blocked with 2 ug anti-CD3CH2CH3 antibody for 30 min at 4° C. in FACS buffer, washed twice with FACS buffer, incubated with 50 ul monomeric single chain anti-CD28/CD3 bispecific antibody supernatant for 30 min at 4° C. in FACS buffer, washed twice with FACS buffer, stained with anti-His Tag PE-conjugated Antibody (R&D System, cat #IC050P), washed three times with FACS buffer and then subjected for FACS analysis. Flow cytometry was performed on a FACS-Calibur apparatus; the CellQuest software was used to acquire and analyze the data (BD BioSciences, Mountain View, Calif.). As shown in FIG. 3B, monomeric single chain anti-CD28/CD3 bispecific antibody had specific binding activities to CD28. FIG. 3A and FIG. 3B data proved that anti-CD28/CD3 bispecific antibody were able to bind to both CD28 and CD3, anti-CD28/CD3 bispecific antibody were a bispecific antibody.

### Activation and Expansion of T Cells Using Monomeric Single Chain Anti-CD28/CD3 Bispecific Antibody

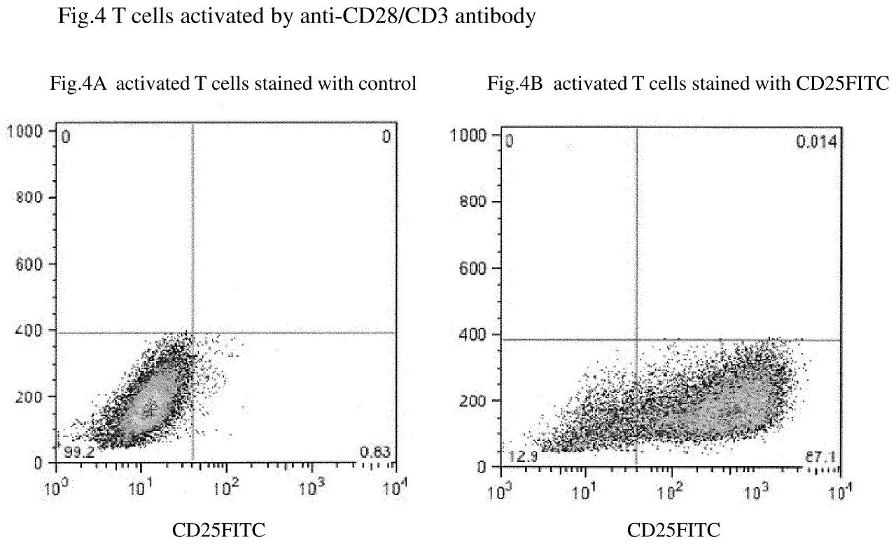

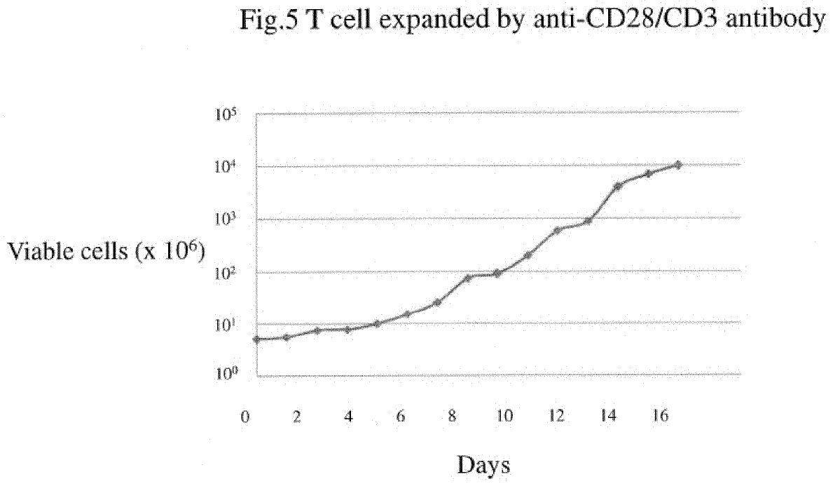

Peripheral blood mononuclear cell (PBMC) was isolated by Ficoll (GE Healthcare Life Sciences) gradient centrifugation of buffy coats from healthy donor. 7×10e6 isolated PBMC was incubated with 2 ml monomeric single chain anti-CD28/CD3 bispecific antibody supernatant for 15 minutes, washed twice with PBS and then cultured in CTS™ OpTmizer™ T Cell Expansion SFM medium (cat #: A1048501, Gibco) supplemented with CTS™ Immune Cell SR (cat #: A2596101), 26 ml CTS™ OpTmizer™ T-Cell Expansion Supplement, 1×L-glutamine (200 mM), 1× PenStrep (Gibco) and 1000 international units IL-2/mL (Cat #200-02, Peprotech) up to 16 days. The culture media were changed every three days. As shown in FIG. 4, 87.1% PBMC was activated by monomeric single chain anti-CD28/CD3 bispecific antibody according to the CD25 expression three days later after incubation. CD25 is one of the T cell activation markers (Shipkova M, 2012). As shown in FIG. 5, PBMC can be efficiently expanded by monomeric single chain anti-CD28/CD3 bispecific antibody according to 16 days cell number counting.

T cells activated by monomeric single chain anti-CD28/CD3 bispecific antibody can be efficiently transduced by lentivirus

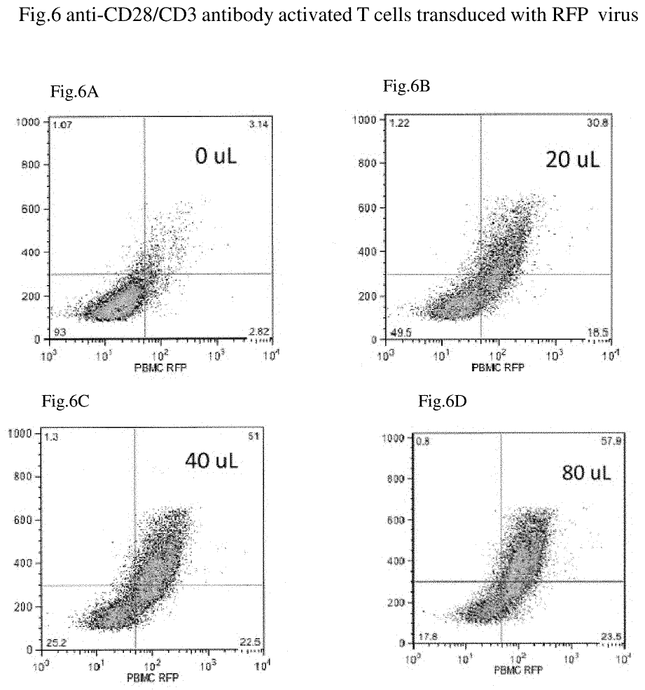

2×10e6 PBMC was incubated with 200 ul monomeric single chain anti-CD28/CD3 bispecific antibody supernatant for 15 minutes, washed two times and then cultured in CTS™ OpTmizer™ T Cell Expansion SFM medium (cat #: A1048501, Gibco) supplemented with CTS™ Immune Cell SR (cat #: A2596101), 26 ml CTS™ OpTmizer™ T-Cell Expansion Supplement, 1×L-glutamine (200 mM), lx PenStrep (Gibco) and 1000 international units IL-2/mL (Cat #200-02, Peprotech) for three days. The T cells activated by monomeric single chain anti-CD28/CD3 bispecific antibody were then transduced by a RFP lentivirus (Cat #pLV-RP, Biosettia CA) according to the user protocol. As shown in FIG. 6, T cells activated by monomeric single chain anti-CD28/CD3 bispecific antibody were transduced even with 20 ul unpurified RFP lentivirus supernatant, which indicated that monomeric single chain anti-CD28/CD3 bispecific antibody activated T cells were highly transducible.

### REFERENCES

Kamphorst A O, Ahmed R.CD4 T-cell immunotherapy for chronic viral infections and cancer. Immunotherapy. 2013 September; 5(9):975-87.
Kalos M, Levine B L, Porter D L, Katz S, Grupp S A, Bagg A, June C H. T cells with chimeric antigen receptors have potent antitumor effects and can establish memory in patients with advanced leukemia. Sci Transl Med. 2011 Aug. 10; 3(95):95ra73.
Maus M V, Grupp S A, Porter D L, June C H. Antibody-modified T cells: CARs take the front seat for hematologic malignancies. Blood. 2014 Apr. 24; 123(17):2625-35.
Xiuyan Wang and Isabelle Rivière. Clinical manufacturing of CAR T cells: foundation of a promising therapy. Mol Ther Oncolytics. 2016; 3: 16015.
Petra Kleindienst and Thomas Brocker. Endogenous Dendritic Cells Are Required for Amplification of T Cell Responses Induced by Dendritic Cell Vaccines In Vivo. J Immunol Mar. 15, 2003, 170 (6) 2817-2823.
T Numbenjapon, L M Serrano, H Singh, C M Kowolik, S Olivares, N Gonzalez, W C Chang, S J Forman, M C Jensen & L J N Cooper. Characterization of an artificial antigen-presenting cell to propagate cytolytic CD19-specific T cells. Leukemia (2006) 20, 1889-1892.
Singh H, Huls H, Kebriaei P, Cooper U. A new approach to gene therapy using Sleeping Beauty to genetically modify clinical-grade T cells to target CD19. Immunol Rev. 2014 January; 257(1):181-90.
Walker M R, Kasprowicz D J, Gersuk V H, Benard A, Van Landeghen M, Buckner J H, Ziegler S F. Induction of FoxP3 and acquisition of T regulatory activity by stimulated human CD4+CD25-T cells. J Clin Invest. 2003 November; 112(9):1437-43.
Geppert T D, Davis L S, Gur H, Wacholtz M C, Lipsky P E. Accessory cell signals involved in T-cell activation. Immunol Rev. 1990 October; 117:5-66.
Peter A. Bretscher. A two-step, two-signal model for the primary activation of precursor helper T cells. Proc Natl Acad Sci USA. 1999 Jan. 5; 96(1): 185-190.
Bretscher P, Cohn M. A theory of self-nonself discrimination. Science. 1970 Sep. 11; 169(3950):1042-9.
Shipkova M, Wieland E. Surface markers of lymphocyte activation and markers of cell proliferation. Clin Chim Acta. 2012 Sep. 8; 413(17-18):1338-49.

Monomeric single chain anti-CD28/CD3 bispecific
antibody DNA sequence
SEQUENCE ID NO 1
ATG GGA TGG TCA TGT ATC ATC CTT TTT CTA GTA GCA ACT
GCA ACC GGT GTA CAT TCC GAC ATC CAG ATG ACC CAG TCT
CCA TCC GCT CTG TCT GCA TCC GTT GGA GAC AGA GTT ACC
ATC ACT TGC CAT GCC AGT CAA AAC ATT TAT GTT TGG TTA
AAC TGG TAC CAG CAG AAA CCA GGA AAA GCC CCT AAA CTC
TTG ATC TAT AAG GCT TCC AAC CTG CAC ACA GGC GTC CCA
TCA AGG TTT AGT GGC AGT GGA TCT GGA ACA GAC TTC ACA
TTA ACC ATC AGC AGC CTG CAG CCT GAA GAC TTT GCC ACT
TAC TAC TGT CAA CAG GGT CAA ACT TAT CCG TAC ACG TTC
GGA GGG GGG ACC AAG GTG GAA ATA AAA GGA GGA GGA GGA
AGC CAG GTC CAA CTG GTG CAG TCC GGA GCT GAG GTG AAG
AAG CCG GGG GCT TCA GTG AAG GTT TCC TGC AAG GCT TCT
GGC TAC ACC TTC ACA AGC TAC TAT ATA CAC TGG GTG AGA
CAG GCG CCT GGA CAG GGA CTT GAG TGG ATT GGA TGT ATT
TAT CCT GGA AAT GTC AAT ACT AAC TAT AAT GAG AAG TTC
AAG GAC AGG GCC ACA CTG ACT GTA GAC ACA TCC ATC AGC
ACT GCC TAC ATG GAG CTC AGC AGA CTG CGC TCT GAC GAC
ACT GCG GTC TAT TTC TGT ACA AGA TCA CAC TAC GGC CTC
GAC TGG AAC TTC GAT GTC TGG GGC CAA GGG ACC ACG GTC
ACC GTC TCC TCA GGT GGT GGT GGT AGT GAT ATC AAA CTG
CAG CAG TCA GGG GCT GAA CTG GCA AGA CCT GGG GCC TCA
GTG AAG ATG TCC TGC AAG ACT TCT GGC TAC ACC TTT ACT
AGG TAC ACG ATG CAC TGG GTA AAA CAG AGG CCT GGA CAG
GGT CTG GAA TGG ATT GGA TAC ATT AAT CCT AGC CGT GGT
TAT ACT AAT TAC AAT CAG AAG TTC AAG GAC AAG GCC ACA
TTG ACT ACA GAC AAA TCC TCC AGC ACA GCC TAC ATG CAA
CTG AGC AGC CTG ACA TCT GAG GAC TCT GCA GTC TAT TAC
TGT GCA AGA TAT TAT GAT GAT CAT TAC TGC CTT GAC TAC
TGG GGC CAA GGC ACC ACT CTC ACA GTC TCC TCA GTC GAA
GGT GGA AGT GGA GGT TCT GGT GGA AGT GGA GGT TCA GGT
GGA GTC GAC GAC ATT CAG CTG ACC CAG TCT CCA GCA ATC
ATG TCT GCA TCT CCA GGG GAG AAG GTC ACC ATG ACC TGC
AGA GCC AGT TCA AGT GTA AGT TAC ATG AAC TGG TAC CAG
CAG AAG TCA GGC ACC TCC CCC AAA AGA TGG ATT TAT GAC
ACA TCC AAA GTG GCT TCT GGA GTC CCT TAT CGC TTC AGT
GGC AGT GGG TCT GGG ACC TCA TAC TCT CTC ACA ATC AGC
AGC ATG GAG GCT GAA GAT GCT GCC ACT TAT TAC TGC CAA
CAG TGG AGT AGT AAC CCG CTC ACG TTC GGT GCT GGG ACC
AAG CTG GAG CTG AAA GAA TTC CAT CAT CAC CAC CAT CAC
TGA

1-2. (canceled)

3. A monomeric single chain bispecific antibody, comprising a single polypeptide having a first and a second antigen binding domain linked by a short peptide, wherein the monomeric single chain bispecific antibody is used for activating T cells, wherein the first antigen binding domain binds to a first antigen on the T cells, providing a primary stimulatory signal for activating the T cells, and the second antigen binding domain binds to a second antigen on the T cells, providing a co-stimulatory signal for activating the T cells.

4. The monomeric single chain bispecific antibody of claim 3, wherein the antigen binding domain comprises a variable domain of an antibody heavy chain and a variable domain of an antibody light chain linked by a short peptide.

5. The monomeric single chain bispecific antibody of claim 3, wherein the antigen binding domain comprises a single chain variable fragment of an antibody (Fv).

6. The monomeric single chain bispecific antibody of claim 3, wherein the first antigen binding domain binds to an antigen selected from a group consisting of T cell receptor, CD3 and CD2.

7. The monomeric single chain bispecific antibody of claim 3, wherein the second antigen binding domain binds to an antigen selected from a group consisting of CD28 and CD9, CD137, ICOS and CD40.

8. The monomeric single chain bispecific antibody of claim 3, wherein the antigen binding domain comprises a ligand that binds to an antigen on the T cells or an antigen-binding fragment of the ligand that binds to an antigen on the T cells.

9. The monomeric single chain bispecific antibody of claim 3, wherein the antigen binding domain is derived from an antibody of a hybridoma clone.

10. The monomeric single chain bispecific antibody of claim 3, wherein the antigen binding domain is derived from a humanized non-human antibody.

11. The monomeric single chain bispecific antibody of claim 3, further comprises a tag domain that facilitates its purification and detection.

12. The monomeric single chain bispecific antibody of claim 3, wherein the T cells activated by the monomeric single chain bispecific antibody may be obtained from a patient or an existing T cell culture.

13. A method for activating T cells using the monomeric single chain bispecific antibody of claim 1, comprising contacting T cells with the monomeric single chain bispecific antibody to activate the T cells.

14. The method for activating T cells of claim 13, further comprising expanding the T cells activated by the monomeric single chain bispecific antibody.

15. T cells that are activated by the method of claim 13.
